SEQUENCE LISTING (4 sequences)

SEQ ID NO: 1 - protein, 397 aa, Homo sapiens
DGILGRDTLPHEDQGKGRQLHSLTLASINTDFTLSLYKKLALRNPDKNVVFSPLSISAALAILSLGAKDSTMEEILEVLKFNLTEITEEEIHHQGFGHLLQRLSQPEDQAEINTGSALFIDKEQPILSEFQEKTRALYQAEAFVADFKQCNEAKKFINDYVSNQTQGKIAELFSELDERTSMVLVNYLLFKGKWKVPFNPNDTFESEFYLDEKRSVKVPMMKIKDLTTPYIRDEELSCSVLELKYTGNASALFILPDQGKMQQVESSLQPETLKKWKDSLRPRIISELRMPKFSISTDYNLEEVLPELGIRKIFSQQADLSRITGTKNLHVSQVVHKAVLDVDETGTEGAAATAVTAALKSLPQTIPPLNFNRPFMLVITDNNGQSVFFMGKVTNPM

SEQ ID NO: 2 - DNA, 1668 bp, Homo sapiens
aacacaccagggcaccctgaacatcaggagtcggcaaacacagaggctagtagctggctggtatcacctctgcagccaggaggacagagaagatggccttcattgcagctttggggctcttgatggcagggatctgccctgctgtcctctgtgatggcatactgggaagggacactctaccccatgaagaccaaggcaaggggagacaactgcacagtctcacattggcttccatcaacactgacttcacattgagcctctacaagaagctggctttgaggaatccagataaaaatgttgtcttctcccctcttagcatctcagctgccttggccattttgtctctgggagcaaaagacagcaccatggaagagattctagaagtactcaagttcaatctcacagagatcactgaggaagaaatccaccaccagggctttgggcacctcctacagaggctcagccagccagaggaccaggcagagatcaatacaggtagtgccctgtttattgacaaagagcagccgatactgtcagaattccaggagaagacaagggctctgtaccaggctgaggccttcgtagctgacttcaagcagtgcaatgaggccaaaaagttcatcaatgactatgtgagcaatcagacccaggggaagatcgcagaactgttctcagaactggatgagaggacatccatggtgctggtgaactatctcctctttaaaggcaaatggaaggtaccatttaaccccaatgacacatttgagtctgagttctacttggatgagaagaggtctgtgaaggtgcccatgatgaaaattaaggatctcaccacaccctacatccgggatgaggagctgtcctgctctgtgctggaactgaagtacacaggaaatgccagcgccctgtttatcctccctgaccagggcaagatgcagcaggtggaatccagcttgcaaccagagaccctgaagaagtggaaggactctctgaggcccaggattataagtgagcttcgcatgcccaagttctccatctccacagactacaacctggaggaggtccttccagagctgggcattaggaaaatcttctcccagcaagctgatctgagtaggatcacagggaccaagaacctgcatgtctctcaggtggtccacaaagctgtgctggatgtggatgagacaggcacagaaggagccgctgccacagcagtcacagcagccctaaaaagtttaccgcaaactatacctcctctgaatttcaaccggccattcatgctggttatcactgacaataatggtcagtctgtcttctttatgggcaaagtcactaaccccatgtgagtctgaagctccccaaaatctgacaattctgcccaggatcctggaacagagcctggatgctgatctctgtatatgccctgacatacatgctctgattggctattgcaaagttggcttagacagtgacatcaactatctctatggctcccatgtgcactggagcctttggattgtcagtgtcaggcacttaggacccttgggagcatctacacatgtttctgaacttggaatctttctttattcttcttccctggtgactcctctttctgtgttcataccccaaaccaagccattgataagtcccagtaaaggttctgagaac

SEQ ID NO: 3 - DNA, 27 bp, artificial sequence
  5′ PCR primer for rat kallistatin
gtcggatcctgatggcatactgggaag

SEQ ID NO: 4 - DNA, 27 bp, artificial sequence
  3′ PCR primer for rat kallistatin
gtggagctcatggggttagtgactttg

(19) United States
(12) Patent Application Publication
(10) Pub. No.: US 2006/0189534 A1
(43) Pub. Date: Aug. 24, 2006

(54) Compounds useful in inhibiting vascular leakage, inflammation and fibrosis and methods of making and using same

(76) Inventor: Jian-xing Ma, Oklahoma City, OK (US)

Correspondence Address: DUNLAP, CODDING & ROGERS P.C., PO BOX 16370, OKLAHOMA CITY, OK, 73113, US

(21) Appl. No.: 11/397,286

(22) Filed: Apr. 4, 2006

Related U.S. Application Data
(63) Continuation of application No. 11/010,794, filed on Dec. 13, 2004, now abandoned.
(60) Provisional application No. 60/528,664, filed on Dec. 11, 2003.

(51) Int. Cl. A61K 38/17

(52) U.S. Cl. 514/12

(57) ABSTRACT

The present invention is directed to a method of inhibiting at least one of vascular leakage, angiogenesis, inflammation and fibrosis in an animal by administering to the animal an effective amount of a composition, wherein the composition is selected from the group consisting of kallistatin, fragments of kallistatin, analogs or derivatives of kallistatin, and combinations thereof.

### CROSS REFERENCE TO RELATED APPLICATIONS

This application is a continuation of U.S. Ser. No. 11/010,794, filed Dec. 13, 2004; which claims benefit under 35 U.S.C. 119(e) of provisional application U.S. Ser. No. 60/528,664, filed Dec. 11, 2003, the contents of which are hereby expressly incorporated herein by reference.

### STATEMENT REGARDING FEDERALLY SPONSORED RESEARCH OR DEVELOPMENT

Not applicable.

### BACKGROUND OF THE INVENTION

1. Field of the Invention

The present invention relates, in general, to compounds useful for inhibiting at least one of vascular leakage, inflammation and fibrosis and methods of making and using same. More particularly, but not by way of limitation, the present invention relates to compounds that are capable of inhibiting at least one of vascular leakage, inflammation and fibrosis in patients (broadly, an animal and more particularly, a mammal or human) that have pathologic conditions exhibiting vascular leakage, inflammation and fibrosis.

2. Background of the Invention

Breakdown of the blood-retinal barrier (BRB), increased vascular permeability and vascular leakage are early complications of diabetes and a major cause of diabetic macular edema (Cunha-Vaz et al., 1985; and Yoshida et al., 1993). At early stages of diabetic retinopathy, it has been determined that the increase of retinal vascular permeability precedes the appearance of clinical retinopathy (Cunha-Vaz et al., 1985; and Yoshida et al., 1993). As there is no satisfactory, non-invasive therapy, diabetic macular edema is a major cause of vision loss in diabetic patients (Moss et al., 1998). Although the pathogenic mechanism underlying the breakdown of the blood-retinal barrier and the increase of retinal vascular permeability is uncertain, the over-production of VEGF (Vascular Endothelial Growth Factor) in the retina is believed to play a key role in the development of vascular hyper-permeability in diabetes (Murata et al., 1996; and Hammes et al., 1998).

VEGF is also referred to as vascular permeability factor (VPF) based on its potent ability to increase vascular permeability (Dvorak et al., 1995; and Aiello et al., 1997). It has been identified as a major causative factor in retinal vascular hyper-permeability (Aiello et al., 1997). The over-expression of VEGF or its receptors is associated with an increased vascular permeability in the retina of streptozotocin (STZ)—induced diabetes (Qaum et al., 2001). There are two possible mechanisms responsible for VEGF—induced vascular hyper-permeability. First, VEGF may act directly on the tight junction of endothelial cells, as it has been shown that VEGF alters the tight junction proteins such as the phosphorylation of occludin and ZO-1 (Antonetti et al., 1999). Second, VEGF may act through the leukocyte-endothelial cell interaction which can trigger endothelial cell adherence and tight junction disorganization (Del Maschio et al., 1996; and Bolton et al., 1998). VEGF has been shown to increase leukocyte stasis through the up-regulation of intercellular adhesion molecule-1 (ICAM-1) (Miyamoto et al., 2000), suggesting that VEGF is also an inflammatory factor. Over-production of VEGF in diabetic retina is believed to be the major cause of vascular leakage, leukostasis and retinal edema, as well as retinal neovascularization in diabetic retinopathy (Aiello et al., 2000).

Diabetic nephropathy (DN) is another one of the most important microvascular complications of diabetes, and DN occurs in 30-40% of diabetic patients (Raptis et al., 2001; and American Diabetes Assoc., 2000). The early changes in DN are characterized by thickening of the glomerular basement membrane and expanded extracellular matrix (ECM), leading to glomerular hyper-filtration and microalbuminuria, renal inflammation and glomerular fibrosis (Raptis et al., 2001; and Sakharova et al., 2001). Although intensified control of hyperglycemia, blood pressure and hyperlipidemia reduces the risks of DN, it does not sufficiently prevent diabetic patients with microalbuminuria from progressing to devastating overt DN, a leading cause of end-stage renal diseases (American Diabetes Assoc., 2000; Anonymous, 1995; and Anonymous, 2000). The exact pathogenesis of DN remains largely unknown.

As with diabetic retinopathy, several growth factors have been suggested to be involved in the pathogenesis of DN, most importantly, transforming growth factor-β (TGF-β) and vascular endothelial growth factor (VEGF) (Chiarelli et al., 2000; and Cooper et al., 2001). TGF-β has been recognized as a modulator of ECM formation. Over-expression of TGF-β in diabetic glomeruli is believed to contribute to matrix accumulation by increasing synthesis and decreasing degradation of extracellular proteins such as fibronectin, leading to glomerular fibrosis (Goldfarb et al., 2001; Greener, 2000; Ng et al., 2003; and Tamaki et al., 2003). Accumulating evidence indicates that VEGF and TGF-β are key pathogenic factors in early stages of DN (Iglesias-de la Cruz et al., 2002; Gambaro et al., 2000; Lane et al., 2001; Kim et al., 2003; Senthil et al., 2003; and Bortoloso et al., 2001). Serum and urinary TGF-β levels have been found to correlate with the severity of microalbuminuri (Pfeiffer et al., 1996; and Ellis et al., 1998). Therefore the increase of the systemic TGF-β levels has been suggested as a marker for DN (Mogyorosi et al., 2000).

Angiogenesis in the retina is controlled by a delicate balance between angiogenic stimulators (e.g., vascular endothelial growth factor—VEGF) and angiogenic inhibitors (e.g., pigment epithelium-derived factor—PEDF) (Jimenez et al., 2001; Bussolino, 1997). Under certain pathological conditions such as diabetic retinopathy and retinopathy of prematurity (ROP), the retinal cells increase the production of angiogenic stimulators while decreasing angiogenic inhibitors in response to local hypoxia (Pierce, 1995; Gao, 2001). These changes break the balance in angiogenesis control and consequently, resulting in over-proliferation of capillary endothelial cells and retinal neovascularization which is a common cause of blindness (Miller, 1997; Jimenez et al., 2001; Blom et al., 1994). The molecular mechanism leading to retinal neovascularization is presently uncertain.

It has been shown that the retina and vitreous fluid contain endogenous angiogenic inhibitors (Preis et al, 1977; Lutty et al., 1983; Lutty et al., 1985; Jacobson et al., 1984; Raymond et al., 1982). PEDF, a serine proteinase inhibitor (serpin), has been identified as a potent angiogenic inhibitor endogenously expressed in the retina (Dawson et al., 1999). Angiostatin has also been identified in human vitreous fluids (Spranger et al., 2000). Decreased levels of angiostatin and PEDF have been shown to correlate with the development of proliferative diabetic retinopathy (Spranger et al., 2000; Spranger et al., 2001).

The tissue kallikrein-kinin system consists of tissue kallikrein, kallikrein-binding protein (also referred to as kallistatin or KBP), kinins, kininogens (precursors of kinins), kininases and bradykinin receptors (Bhoola et al., 1992). Tissue kallikrein is a serine proteinase which cleaves kininogens to release vasoactive kinins. Kinins interact with bradykinin receptors on the cell surface and exert a variety of biological effects. It is known that most functions of kinins such as vasodilation, regulation of local blood flow and tissue metabolic rate, production of pain and inflammatory responses, are mediated by the B2 kinin receptor (Bhoola et al., 1992; Schachter, 1983). Kinins also have a direct mitogenic effect on endothelial cells (Bhoola et al., 1992; Schachter, 1983). It has been shown recently that the angiogenic activity of kinins is mediated by the B1 kinin receptor (Hu et al., 1993; Emanueli et al, 2002).

Kallistatin was originally identified from rat serum as it binds to tissue kallikrein, forming a SDS-stable complex (Chao et al., 1986; Chao et al., 1990). It inhibits the proteolytic activity of kallikrein in a transgenic mouse over-expressing kallikrein. Recently, kallistatin has been shown to have vascular function independent of its interactions with the kallikrein-kinin system (Chao et al., 2001; Miao et al., 2002).

Kallistatin is a glycoprotein of 425 amino acids and having a molecular weight of 58 kDa. Kallistatin is predominantly produced in the liver, and it has also been identified in a number of other tissues including the retina and vitreous (Hatcher et al., 1997). Kallistatin shares significant sequence homology with other serpins such as α1-antitrypsin, α1-antichymotrypsin and PEDF, suggesting that it belongs to the serpin super family (Chai et al., 1991). Like many other serpins, kallistatin specifically binds to heparin.

The serpin super family consists of multiple proteins with widely diverse functions (Silverman et al., 2001). Some of the serpin members, such as PEDF, antithrombin and maspin, have been shown to have anti-angiogenic activity (Dawson et al., 1999; O'Reilly et al., 1999; Zhang et al., 2000). Previous evidence indicates that kallistatin is involved in blood pressure regulation, inflammatory response and animal growth (Yoon et al., 1987; Ma et al., 1995; Hatcher et al., 1999). In ocular tissues, kallistatin levels were reduced in the retina of rats with streptozotocin (STZ)—induced diabetes and in vitreous from patients with proliferative diabetic retinopathy (Hatcher et al., 1997; Ma et al., 1996). These results suggest that kallistatin has certain functions independent of its interactions with the kallikrein-kinin system (Chen et al., 1996).

There is currently a need in the art for new methods of specifically inhibiting angiogenesis, vascular leakage, inflammation and fibrosis that are effective and substantially non-toxic to the animal suffering from pathologic vascular leakage, inflammation and fibrosis. It is to such methods that the presently disclosed and enabled invention are directed.

### SUMMARY OF THE INVENTION

According to the present invention, methods of inhibiting at least one of vascular leakage, inflammation and fibrosis are provided. Broadly, the present invention is related to a new function that has been discovered for kallistatin, a serine protease known to bind tissue kallikrein and regulate blood pressure. The methods of the present invention involve administration of a composition capable of inhibiting at least one of vascular leakage, inflammation and fibrosis to an animal, in need thereof, wherein the composition is selected from the group consisting of kallistatin, fragments of kallistatin, analogs or derivatives of kallistatin, and combinations thereof.

It is an object of the present invention to provide a method of inhibiting at least one of vascular leakage, pathological angiogenesis, inflammation and fibrosis in an animal (such as a mammal or human) suffering from pathologic vascular leakage, cancer, inflammation and/or fibrosis or having a predisposition for vascular leakage, cancer, inflammation and/or fibrosis. The method includes administering to the animal an effective amount of the composition described herein above. The animal experiencing the pathologic condition may have a disease (or be predisposed to a disease) selected from the group consisting of diabetes, chronic inflammation, brain edema, edema, arthritis, uvietis, ascites, macular edema, cancer, hyperglycemia, a kidney inflammatory disease, a disorder resulting in kidney fibrosis, a disorder of the kidney resulting in proteinuria, and combinations thereof.

It is a further object of the present invention, while achieving the before-stated object, to provide a composition having an activity that inhibits at least one of vascular leakage, inflammation and fibrosis and an activity that inhibits pathological angiogenesis. A substantially higher amount of the composition must be administered to an animal for the composition to exhibit the inhibition of angiogenesis activity, whereas a substantially lower amount of the composition exhibits the activity that inhibits at least one of vascular leakage, inflammation and fibrosis when administered to an animal.

Other objects, features and advantages of the present invention will become apparent from the following detailed description when read in conjunction with the accompanying drawings and appended claims.

### BRIEF DESCRIPTION OF THE SEVERAL VIEWS OF THE DRAWINGS

FIG. 1 illustrates the expression and purification of recombinant kallistatin. (a) SDS-PAGE with Coomassie blue staining; (b) Western blot analysis with an antibody specific to the His-tag. Lane 1, crude cell extract before IPTG induction; 2, crude extract after IPTG induction and 3, affinity-purified kallistatin.

FIG. 2 illustrates the effect of kallistatin on cell viability. Primary RCEC, pericytes and the Müller cell line were treated with recombinant kallistatin at concentrations as indicated for 72 h. The viable cells were quantified using the MTT assay. Values represent absorbance as percentages of respective controls (means±SD, n=3), and * indicates the values statistically different from the control (P<0.05).

FIG. 3 illustrates inhibition of [³H] thymidine incorporation by kallistatin in endothelial cells. RCEC were treated with kallistatin, and the effect on proliferation rate was determined by [³H] thymidine incorporation assay. Bars represent [³H] incorporated into the chromosome (mean±SD, n=4) and values statistically different from the control are indicated by * (P<0.05).

FIG. 4 illustrates quantitative analysis of apoptosis induced by kallistatin in endothelial cells. RCEC were treated with different concentrations of kallistatin for 24 h and stained with Annexin V and PI. Apoptotic cells were quantified by flow cytometry. (a) cytograms from flow cytometric analysis. Intact cells, early apoptotic cells, and late apoptotic and necrotic cells are located in the lower left, lower right, and upper right quadrants of the cytograms, respectively. (b) percentages of early apoptotic cells (means±SD, n=4). 1, control RCEC; 2, RCEC treated with colchicine as positive control; 3, 4, 5 and 6, RCEC treated with 40, 160, 320 and 640 nM of kallistatin, respectively. Values significantly higher than control (P<0.05) are indicated by *.

FIG. 5 illustrates inhibition of ischemia-induced retinal neovascularization by intravitreal injection of kallistatin. Retinal neovascularization was induced in newborn Brown Norway rats. Retinal vasculature was examined by angiography at 5 days after the injection of kallistatin or PBS (control). (a) retina from OIR rats after PBS injection; (b) retina from OIR rats after kallistatin injection; (c) retina from age-matched normal rats after PBS injection and (d) retina from normal rats after kallistatin injection. Each image is a representative from 4 animals of each group. (e) Pre-retinal vascular cells were counted on saggital sections from 8 animals. Bars represent cell average numbers per section (mean±SD, n=8). The number in each kallistatin-treated group was compared with the control by Student's t test and * indicates the group with statistical difference from the control (P<0.05).

FIG. 6 illustrates kallistatin dose-dependent reduction of vascular leakage. Rats with OIR received an intravitreal injection of 3 ml of kallistatin at P14. Permeability was measured at P16. Evans blue-albumin leakage was normalized by total protein concentration and expressed as microgram of Evans blue per milligram total protein (mean±SD, n=4). (a) retina; (b) iris; (c) chorid. 1, age-matched normal rats injected with PBS; 2, OIR rats with PBS injection; 3, 4 and 5, OIR rats injected with 2.4, 4.8 and 9.6 mg/ml kallistatin, respectively. Values with statistical difference from the PBS-injected OIR control are indicated by *.

FIG. 7 illustrates the effects of the B1 and B2 receptor antagonists on RCEC proliferation. RCEC were separately treated with kallistatin, the B1 receptor antagonist and B2 receptor antagonist for 48 h and viable cells quantified by MTT assay. Viable cell numbers are expressed as percentages of the control (mean±SD, n=4). 1, control cells treated with PBS; 2, 40 nM kallistatin alone; 3, 5 mM of the B1 antagonist and 4, 5 mM of the B2 antagonist.

FIG. 8 illustrates inhibition of VEGF binding to RCEC by kallistatin. ¹²⁵I-VEGF was incubated with RCEC in the absence and presence of excess amounts of kallistatin or K5 as indicated. The binding of VEGF on RCEC was measured. Bars represent the bound VEGF (CPM) per well (mean±SD, n=3) and * indicates the values statistically different from the control (VEGF alone) (P<0.05).

FIG. 9 illustrates down-regulation of VEGF expression by kallistatin in RCEC and in the retina. RCEC were treated with various concentrations of kallistatin under hypoxia for 24 h. The conditioned medium and cells were separately harvested for VEGF measurements. (a) kallistatin decreased VEGF levels in the conditioned medium. VEGF levels in the conditioned medium were measured by ELISA, normalized by total protein concentrations in the medium and expressed as picogram of VEGF per milligram of total protein (mean±SD, n=4). 1, medium from normoxic culture; 2, medium from hypoxic culture; 3, 4, 5, 6 and 7, medium from hypoxic culture treated with 5, 10, 20, 40 and 80 nM kallistatin, respectively. (b) kallistatin decreased cellular VEGF levels in RCEC. VEGF levels in cell lysates were measured by Western blot analysis, semi-quantified by densitometry and normalized by β-actin level. The relative VEGF levels were expressed as percentages of that in the control cultured under normoxia (mean±SD, n=3). Lane 1, control cells under normoxia; 2, cells under hypoxia; 3, 4 and 5, cells treated with 40, 160 and 640 nM kallistatin, respectively, under hypoxia. (c) Intravitreal injection of 25 μg kallistatin decreased retinal VEGF levels. Rats with retinal neovascularization were injected with kallistatin or the same volume of PBS (control). Retinal VEGF levels were measured by Western blot analysis, semi-quantified by densitometry, normalized by β-actin and expressed as percentages of the control (mean±SD, n=3). 1, OIR retina with PBS injection and 2, OIR retina with kallistatin injection.

FIG. 10 illustrates the decreased expression of kallistatin in the kidney of a diabetic rat model. Diabetes was induced in Brown Norway rats by an injection of streptozotocin (STZ) and confirmed by blood glucose levels. Six weeks after the onset of diabetes, rats were euthanized. The kidney was dissected and homogenized. Kallistatin levels in the soluble fraction of the kidney homogenates were measured by a specific ELISA and normalized by total protein concentrations (mean±SD, n=5). Kaliistatin levels were significantly lower in diabetic kidney than that in the age-matched control kidney (P<0.01).

FIG. 11 illustrates blockage of high glucose-induced fibronectin secretion by kallistatin in human mesangial cells (HMC). Primary HMC was treated with high glucose (30 mM) in the presence of different concentrations of kallistatin as indicated for 3 days. Control cells were cultured in 5 mM glucose. To overcome the osmolarity difference, mannitol control cells were treated with 25 mM mannitol+5 mM glucose under the same conditions. Fibronectin secreted into the culture medium was measured by ELISA (mean±SD, n=3). High glucose increased fibronectin secretion significantly. Kallistatin displayed a concentration-dependent decrease in fibronectin secretion in high glucose. In all the concentrations of kallistatin with high glucose, fibronectin was significantly lower than the high glucose alone (P<0.01).

FIG. 12 illustrates that kallistatin blocks TGF-β-induced fibronectin over-production in HMC. HMC were treated with 5 ng/ml TGF-β without or with different concentrations of kallistatin for 3 days. Fibronectin secreted into the medium was measured by ELISA (mean±SD, n=3). TGF-β induced significant over-production of fibronectin. Kallistatin blocked the TGF-β-induced fibronectin production in a concentration-dependent manner (P<0.01 in all concentrations of kallistatin).

FIG. 13 illustrates prevention of the high glucose-induced decrease of PEDF in HMC by kallistatin. HMC were treated with high glucose (30 mM) in the presence of different concentrations of kallistatin for 3 days. Control cells were treated with 5 mM glucose and 5 mM glucose+25 mM mannitol as an osmolarity control. PEDF secretion into the medium was measured by ELISA (mean±SD, n=3). High glucose decreased PEDF levels, and kallistatin prevents the decrease of PEDF under the high glucose insult (P<0.05).

### DETAILED DESCRIPTION OF THE INVENTION

Before explaining at least one embodiment of the invention in detail, it is to be understood that the invention is not limited in its application to the details of construction and the arrangement of the components set forth in the following description, the experimental details or results, or illustrated in the appended drawings. The invention is capable of other embodiments or of being practiced or carried out in various ways that would be appreciated by one of ordinary skill in the art as being encompassed by the scope of the presently disclosed and enabled invention. Also, it is to be understood that the phraseology and terminology employed herein is for the purpose of description and should not be regarded as limiting.

Kallistatin is a member of the serpin super family that specifically binds to tissue kallikrein, forming a covalent complex (Chao et al., 1990). The amino acid sequence of kallistatin is shown in SEQ ID NO:1, while the nucleotide sequence encoding kallistatin is shown in SEQ ID NO:2. The present invention has shown that kallistatin inhibited the development of retinal neovascularization and decreased vascular leakage in the retina, iris and choroid in a rat model of OIR. The results of the present invention also showed that kallistatin blocks VEGF binding to its receptors and down-regulates VEGF expression, which may represent a mechanism responsible for its anti-angiogenic activity.

Kallistatin is known to form a covalent complex with tissue kallikrein (Chao et al., 1990). Delivery of the kallistatin gene into a transgenic mouse over-expressing kallikrein reverses the effect of kallikrein on blood pressure regulation, which provides in vivo evidence that kallistatin inhibits the activity of tissue kallikrein, and this inhibition may contribute to the regulation of vasodilation and local blood flow (Ma et al., 1995). Kallistatin is present in the retina and vitreous at high levels, suggesting that it may have physiological functions in the ocular tissues (Hatcher et al., 1997; Ma et al., 1996). The vitreous kallistatin levels were decreased in patients with proliferative diabetic retinopathy, suggesting its possible role in diabetic retinopathy (Ma et al., 1996). The results presented herein revealed new activities for this serpin, including but not limited to, inhibition of angiogenesis, vascular permeability and vascular leakage.

The terms “kallistatin”, “kallikrein-binding protein”, and “KBP” are used herein interchangeably.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)) and Coligan et al. Current Protocols in Immunology (Current Protocols, Wiley Interscience (1994)), which are expressly incorporated herein by reference in their entirety. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or (4) does not occur in nature.

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is referred to herein as “naturally-occurring”.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 60%, and more typically with preferably increasing homologies of at least 65%, 70%, 75%, 80%, 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at least more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, “substantial identity”, “variant” and “ortholog”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions, and truncations in the polypeptide encoded by the reference sequence, as discussed herein.

A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

An “ortholog” denotes a polypeptide or polynucleotide obtained from another species that is the functional counterpart of a polypeptide or polynucleotide from a different species. Sequence differences among orthologs are the result of speciation.

As used herein, the twenty conventional amino acids and their abbreviations follow conventional usage. See Immunology—A Synthesis (2.sup.nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as .alpha.-,.alpha.-disubstituted amino acids, N-alkyl amino acids, lactic acid, and other unconventional amino acids may also be suitable components for polypeptides of the present invention. Examples of unconventional amino acids include: 4-hydroxyproline, .gamma.-carboxyglutamate, .epsilon.-N,N,N-trimethyllysine, .epsilon.-N-acetyllysine, O-phosphoserine, N-acetylserine, N-formylmethionine, 3-methylhistidine, 5-hydroxylysine, .sigma.-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of compositions having inhibition of vascular leakage, inflammation and fibrosis activities are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of proteins or peptides of the present invention can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

“Treatment” refers to both therapeutic treatment and prophylactic or preventative measures. Those in need of treatment include those already with the disorder as well as those in which the disorder is to be prevented.

A “disorder” is any condition that would benefit from treatment with the compositions exhibiting inhibition of at least one of vascular leakage, inflammation and fibrosis activities utilized in accordance with the methods of the present invention. This includes chronic and acute disorders or diseases including those pathological conditions which predispose the mammal to the disorder in question.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hopatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

“Mammal” for purposes of treatment refers to any animal classified as a mammal, including human, domestic and farm animals, nonhuman primates, and zoo, sports, or pet animals, such as dogs, horses, cats, cows, etc. The term “patient” refers to human and veterinary subjects.

The term “effective amount” refers to an amount of a biologically active molecule or conjugate or derivative thereof sufficient to exhibit a detectable therapeutic effect without undue adverse side effects (such as toxicity, irritation and allergic response) commensurate with a reasonable benefit/risk ratio when used in the manner of the invention. The therapeutic effect may include, for example but not by way of limitation, inhibiting permeability of vessels and other vasculature. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy”, and will be understood to mean that the patient in need of treatment is treated or given another drug for the disease in conjunction with the compositions of the present invention. This concurrent therapy can be sequential therapy where the patient is treated first with one drug and then the other, or the two drugs are given simultaneously.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism. A molecule can be biologically active through its own functionalities, or may be biologically active based on its ability to activate or inhibit molecules having their own biological activity.

The compounds of the present invention may be administered to a subject by any method known in the art, including but not limited to, oral, topical, transdermal, parenteral, subcutaneous, intranasal, intramuscular, intraperitoneal, intravitreal and intravenous routes, including both local and systemic applications. In addition, the compounds of the present invention may be designed to provide delayed, controlled or sustained release using formulation techniques which are well known in the art. Such techniques are disclosed in greater detail in Atty Dkt No. 5820.656, filed Dec. 13, 2004, the contents of which are hereby expressly incorporated herein by reference.

The present invention also includes a pharmaceutical composition comprising a therapeutically effective amount of at least one of the compositions described hereinabove in combination with a pharmaceutically acceptable carrier. As used herein, a “pharmaceutically acceptable carrier” is a pharmaceutically acceptable solvent, suspending agent or vehicle for delivering the compounds of the present invention to the human or animal. The carrier may be liquid or solid and is selected with the planned manner of administration in mind. Examples of pharmaceutically acceptable carriers that may be utilized in accordance with the present invention include, but are not limited to, PEG, liposomes, ethanol, DMSO, aqueous buffers, oils, and combinations thereof.

The present invention is related to methods of inhibiting at least one of vascular leakage, angiogenesis, inflammation and fibrosis due to a disease or disorder, such as but not limited to diabetes, by administration of an effective amount of a compound selected from the group consisting of kallistatin, analogs or derivatives of kallistatin, and combinations thereof. Further, one of ordinary skill in the art will appreciate that any compound described herein can be modified or truncated and retain the desired inhibition of at least one of vascular leakage, inflammation and fibrosis activities. As such, active fragments of the compounds described herein are suitable for use in the present inventive methods.

Therefore, the terms “KBP”, “kallistatin” and “kallikrein-binding protein” as used herein will be understood to refer to kallistatin as described herein above, peptide fragments of kallistatin that have at least one of vascular leakage-, angiogenesis-, inflammation- and fibrosis-inhibiting activities; and analogs or derivatives of kallistatin that have substantial sequence homology (as defined herein) to the amino acid sequence of kallistatin which have at least one of vascular leakage-, angiogenesis-, inflammation- and fibrosis-inhibiting activities.

The proteins utilized in accordance with the present invention may be selected from the group consisting of a protein or peptide comprising an amino acid sequence in accordance with SEQ ID NO:1; a protein having at least 60% sequence identity to SEQ ID NO:1; a protein having at least 65% sequence identity to SEQ ID NO:1; a protein having at least 70% sequence identity to SEQ ID NO:1; a protein having at least 75% sequence identity to SEQ ID NO:1; a protein having at least 80% sequence identity to SEQ ID NO:1; a protein having at least 85% sequence identity to SEQ ID NO:1; a protein having at least 90% sequence identity to SEQ ID NO:1; a protein having at least 95% sequence identity to SEQ ID NO:1; a peptide comprising a sequence in accordance with at least a portion of SEQ ID NO:1; a protein or peptide comprising conservative or semi-conservative amino acid changes when compared to SEQ ID NO:1; an ortholog of SEQ ID NO:1; a variant of SEQ ID NO:1; a protein or peptide encoded by at least a portion of the nucleotide sequence in accordance with SEQ ID NO:2; a protein or peptide encoded by a nucleotide sequence which will hybridize to a complementary sequence of SEQ ID NO:2 or a fragment thereof; a protein or peptide encoded by a nucleotide sequence which but for the degeneracy of the genetic code or encoding of functionally equivalent amino acids would hybridize to one of the nucleotides sequences defined immediately herein above. All of the proteins or peptides described immediately herein above must retain the ability to inhibit at least one of angiogenesis, vascular leakage, inflammation and fibrosis.

The kallistatin proteins utilized in accordance with the present invention may be isolated from body fluids, such as but not limited to blood or urine. Optionally, the kallistatin proteins utilized in accordance with the present invention may be synthesized by recombinant, enzymatic or chemical methods. Such recombinant, enzymatic and chemical methods are fully within the skill of a person of ordinary skill in the art, and thus kallistatin proteins produced by such methods are fully within the scope of the present invention. When recombinant methods of producing kallistatin are utilized in accordance with the present invention, the kallistatin may be in a solubilized, refolded form, or the kallistatin may be in the form of an aggregate.

Preferred methods of administration of the compositions described herein above in accordance with the methods of the present invention include oral, topical, transdermal, parenteral, subcutaneous, intranasal, intramuscular, intraperitoneal, intravitreal, intradermal, intraocular, periocular, subconjunctival, retrobulbar, intratracheal, and intravenous routes, including both local and systemic applications. Preparation of a composition for administration by one or more of the routes described herein above are within the skill of a person having ordinary skill in the art, and therefore no further description is deemed necessary.

In addition, the compositions of the present invention may be designed to provide delayed or controlled release using formulation techniques which are well known in the art.

The amount of the compositions of the present invention required to exhibit the inhibition of vascular leakage activity in an animal may be at least 10-fold lower than the amount required to exhibit the anti-angiogenic activity of the composition, and preferably may be at least 50-fold lower than the amount required to exhibit the anti-angiogenic activity of the composition, and more preferably may be at least 100-fold lower than the amount required to exhibit the anti-angiogenic activity of the composition.

Further, the methods of the present invention also envisage administration of an isolated nucleotide sequence, such as a DNA molecule, encoding kallistatin or an enzymatically active variant thereof, a fragment or derivative of kallistatin, or combinations thereof. It is within the skill of a person having ordinary skill in the art to identify and administer DNA molecules that could be utilized in accordance with the present invention.

The invention is further illustrated by the following examples, which are not to be construed in any way as imposing limitations upon the scope of the present invention. On the contrary, it is to be clearly understood that various other embodiments, modifications, and equivalents thereof, after reading the description herein in conjunction with the Drawings and appended claims, may suggest themselves to those skilled in the art without departing from the spirit and scope of the presently disclosed and claimed invention.

### EXAMPLE 1

### Kallistatin Inhibits Retinal Neovascularization and Decreases Vascular Leakage

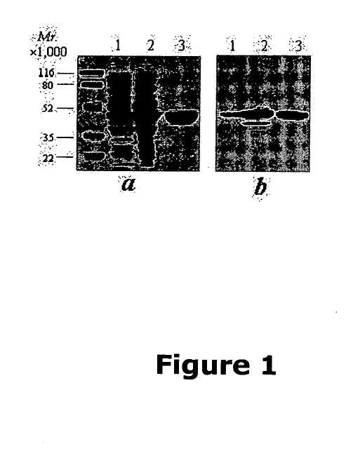

Now referring to the Figures, FIG. 1 illustrates the expression and purification of kallistatin. Kallistatin was expressed in E. coli and purified to apparent homogeneity with the His.Bind affinity column. The purified recombinant protein showed an apparent molecular weight of 45 kDa, matching the calculated molecular weight from the sequence (FIG. 1a). The molecular weight of the recombinant protein is different from native kallistatin (60 kDa) due to the lack of glycosylation in E. coli (Chao et al., 1990). The identity of the band was confirmed by Western blot analysis using an anti-His tag antibody (FIG. 1b). An average of 20 mg of purified kallistatin was obtained from 1 L of culture.

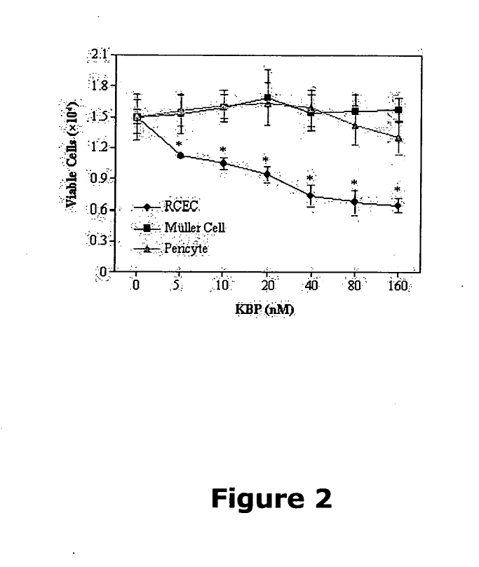

FIG. 2 illustrates the specific inhibition of endothelial cell proliferation by recombinant kallistatin. RCEC were treated with recombinant kallistatin at concentrations of 5, 10, 20, 40, 80 and 160 nM for 72 h. Viable cells were quantified by MTT assay. At a concentration as low as 5 nM, kallistatin treatment resulted in significantly fewer viable cells than the control cells (P<0.05, n=3). This effect appeared to be kallistatin concentration-dependent, with an apparent IC₅₀ of 50 nM (FIG. 2) which is similar to that of K5, a known angiogenic inhibitor (Zhang et al., 2001). At the same concentrations, kallistatin did not result in any significant inhibition of pericytes from the same origin as the RCEC or of the Müller cell line (P>0.05, n=4), suggesting that kallistatin inhibition is specific to endothelial cells (FIG. 2).

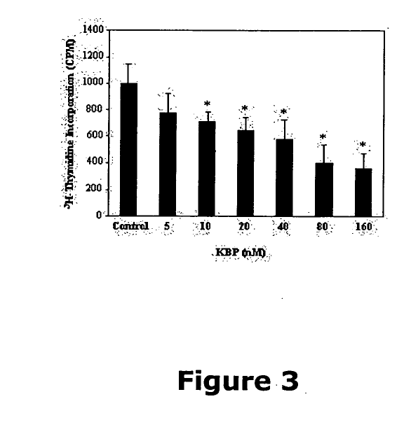

The effect of kallistatin on cell proliferation rate was measured by [³H]-thymidine incorporation assay, as shown in FIG. 3. Kallistatin inhibited thymidine incorporation in RCEC in a concentration-dependent manner from 5 to 160 nM.

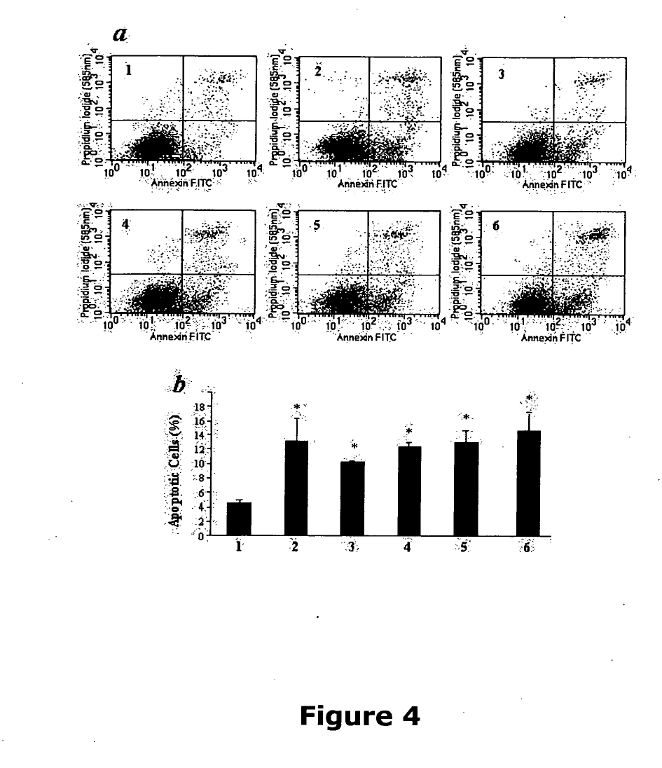

To determine whether kallistatin might induce cell death, RCEC were incubated with different concentrations of kallistatin or 10 μM chochicine for 24 h, and the apoptotic cells were quantified using the Annexin V-flow cytometry method. Phosphatidylserine externalization is a characteristic of cells undergoing apoptosis. The Annexin V-FITC kit allows for fluorescent detection of Annexin V bound to apoptotic cells and quantitative determination by flow cytometry. The Annexin V-FITC kit uses Annexin V conjugated with fluorescein isothiocyante (FITC) to label phosphatidylserine sites on the membrane surface. The kit includes propidium iodide (PI) to label the cellular DNA in necrotic cells where the cell membrane has been totally compromised. This combination allows the differentiation among early apoptotic cells (Annexin V positive, PI negative), necrotic cells (Annexin V positive, PI positive), and viable cells (Annexin V negative, PI negative) which can be located in the lower right, upper right, and lower left quadrants of the cytograms, respectively (FIG. 4a). Because only cells that are Annexin V-positive and PI-negative are truly apoptotic cells, the percentage of this cell population was quantified. The results showed that kallistatin increases apoptosis in RCEC in a dose-dependent manner (FIG. 4).

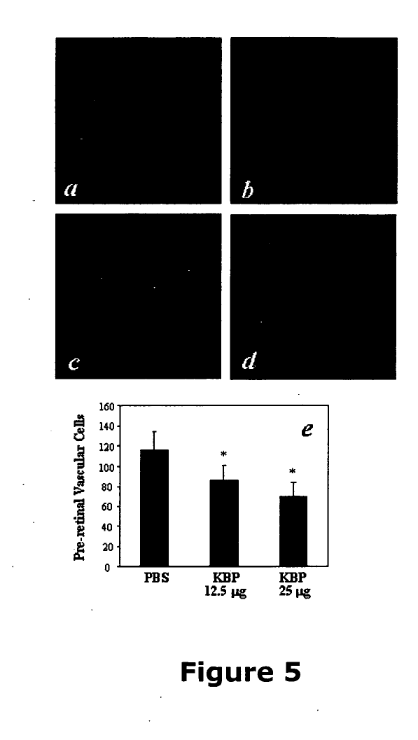

Retinal neovascularization was induced in Brown Norway rats by exposure of newborn rats to hyperoxia as described previously (Gao et al., 2001). Kallistatin was injected intravitreally at P12, and the control eyes received the same volume of PBS. Rats were kept under normoxia for another 5 days, and retinal neovascularization was examined by fluorescein angiography (P17). The control eyes (PBS-injected) developed typical retinal neovascularization, including neovascular tufts, microaneurysms, enlarged non-perfusion regions and vascular leakage (FIG. 5a). Kallistatin injection showed an apparent improvement in retinal vasculature (FIG. 5b). Kallistatin injection did not result in any apparent difference in retinal vasculature of normal rats (FIGS. 5c & d).

Quantification of pre-retinal neovascular cells demonstrated that injection of 12.5 and 25 μg of kallistatin per eye both significantly decreased pre-retinal vascular cells (P<0.01, n=8) (FIG. 5e). This result demontrates that a single kallistatin injection inhibits retinal neovascularization under ischemic conditions.

No apparent histological evidence of retinal toxicity was observed in any analyzed retinal sections after the kallistatin injection (data not shown), suggesting that kallistatin, at the concentrations used, does not cause any detectable toxicity to the retina or to the normal vasculature.

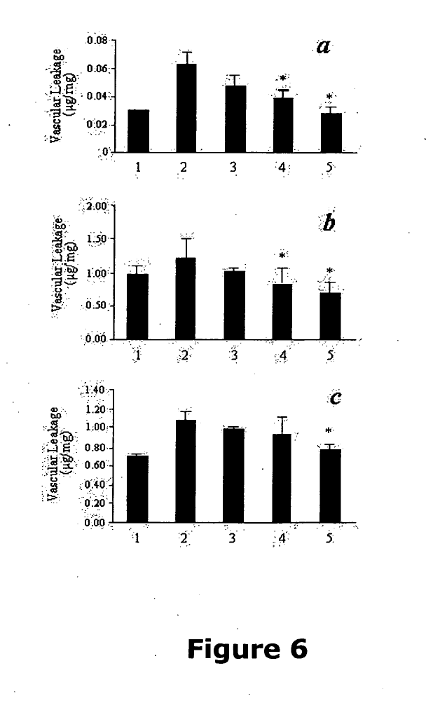

Next, the effect of kallistatin on vascular permeability was determined. At P14, two days after the rats were returned to normoxia, the OIR rats received an intravitreal injection of 3 μl of kallistatin of concentrations of 2.4, 4.8 and 9.6 mg/ml in the right eye (4 animals per dose group), and the same volume of PBS in the left. Two days after the kallistatin injection (P16), vascular permeability was measured by the Evans blue leakage method. The rats exposed to hyperoxia showed significantly increased vascular permeability in the retina, iris and choroid when compared to the age-matched normal rats (FIG. 6). Kallistatin injection decreased vascular permeability in a dose-dependent manner in the retina, iris and choroid of the hyperoxia-treated rats (FIG. 6). At the high dose (9.6 mg/ml), kallistatin injection resulted in a significant decrease in permeability in all three tissues (P<0.01 in the retina and iris, and P<0.05 in the choroid, n=4). At the concentration of 4.8 mg/ml, kallistatin significantly decreased vascular leakage in the retina and iris (P<0.05) but not in the choroid. At 2.4 mg/ml, kallistatin did not show any significant effect in all three tissues (FIG. 6).

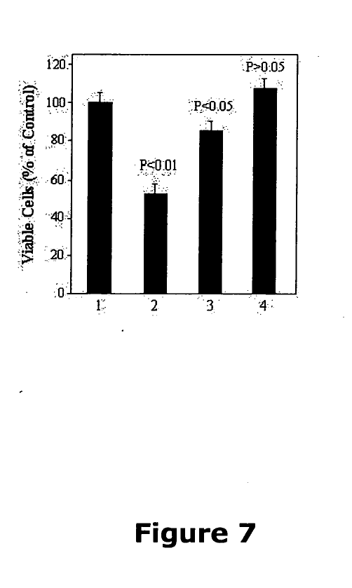

FIG. 7 illustrates the effects of B₁ and B₂ kinin receptor antagonists on RCEC. In order to test whether the anti-angiogenic activity of kallistatin is via reducing kinin production by inhibiting kallikrein activity, RCEC were treated with 5 μM des-Arg⁹-[Leu⁸]-bradykinin, a specific antagonist of the B₁ kinin receptor or Hoe-140, a specific B₂ kinin receptor antagonist, in the presence or absence of 40 nM kallistatin for 48 h, and viable cells were quantified by MTT assay. As shown in FIG. 7, kallistatin treatment resulted in viable cell numbers of approximately 50% of the control (P<0.01, n=4), while the B₁ antagonist treatment resulted in viable cells of 85% of the control (P<0.05, n=4). The B₂ antagonist showed no significant inhibition of RCEC at a high concentration (5 μM)(P>0.05, n=4) (FIG. 7). The complete blockade of the B₁ receptor showed significantly weaker inhibition of RCEC compared to kallistatin alone (P<0.01, n=4), suggesting that the kallistatin-induced inhibition of RCEC cannot be through reducing kinin production.

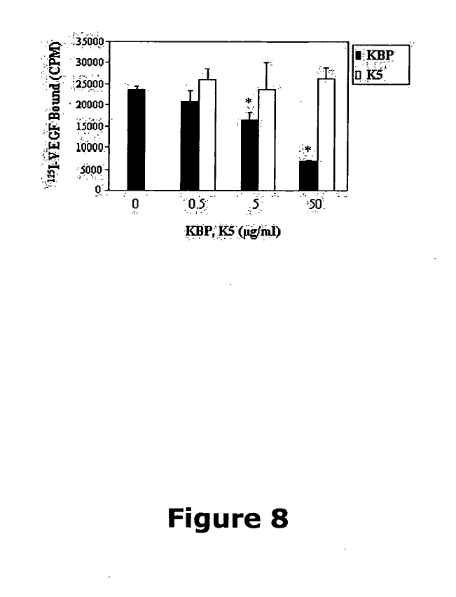

FIG. 8 illustrates inhibition of VEGF binding to RCEC by kallistatin. Incubation of ¹²⁵I-VEGF with RCEC for 1 h resulted in significant binding of VEGF to RCEC. To determine the competition between kallistatin and VEGF in RCEC binding, ¹²⁵I-VEGF was added to RCEC together with 0.5, 5 and 50 μg of unlabeled kallistatin to result in VEGF:kallistatin molar ratios of 1:5, 1:50 and 1:500, respectively. In the presence of excess amounts of kallistatin, VEGF bound to RCEC was decreased in a kallistatin concentration-dependent manner (FIG. 8). In contrast, K5 did not inhibit VEGF binding with RCEC in the same concentration range, suggesting different mechanisms of action between kallistatin and K5 (FIG. 8), although they both specifically inhibit endothelial cells.

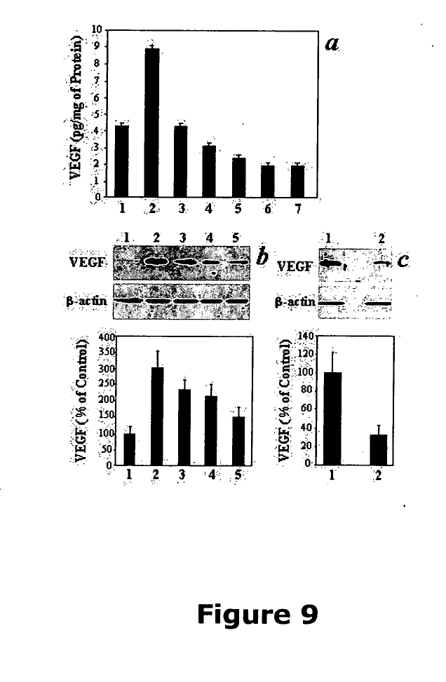

FIG. 9 illustrates down-regulation of VEGF expression by kallistatin. As increased VEGF levels in the retina and vitreous play a key role in the development of retinal neovascularization, the effect of kallistatin on the expression of VEGF in cultured RCEC was determined. VEGF secreted into the conditioned medium was measured by VEGF ELISA and normalized by total protein concentration in the medium. The result showed that kallistatin treatment resulted in reduced VEGF in the medium, and the effect appeared to be kallistatin concentration-dependent (FIG. 9a). Western blot analysis showed that kallistatin also reduced VEGF levels in the cell lysate of RCEC in a concentration-dependent manner (FIG. 9b).

The effect of kallistatin on VEGF expression was also examined in vivo. After intravitreal injection of 25 μg kallistatin, VEGF levels were determined in the retina with OIR. Consistent with the results in cultured RCEC, kallistatin injection decreased retinal VEGF levels to approximate 35% of the control (P<0.01, n=3) (FIG. 9c), suggesting that the vascular activities of kallistatin in this animal model may be through down-regulation of VEGF expression in the retina.

As stated herein above, kallistatin is a member of the serpin super family that specifically binds to tissue kallikrein, forming a covalent complex (Chao et al., 1990). The present invention has shown that kallistatin inhibited the development of retinal neovascularization and decreased vascular leakage in the retina, iris and choroid in a rat model of OIR. The results of the present invention also showed that kallistatin blocks VEGF binding to its receptors and down-regulates VEGF expression, which may represent a mechanism responsible for its anti-angiogenic activity.

kallistatin is known to form a covalent complex with tissue kallikrein (Chao et al., 1990). Delivery of the kallistatin gene into a transgenic mouse over-expressing kallikrein reverses the effect of kallikrein on blood pressure regulation, which provides in vivo evidence that kallistatin inhibits the activity of tissue kallikrein, and this inhibition may contribute to the regulation of vasodilation and local blood flow (Ma et al., 1995). Kallistatin is present in the retina and vitreous at high levels, suggesting that it may have physiological functions in the ocular tissues (Hatcher et al., 1997; Ma et al., 1996). Vitreous kallistatin levels were decreased in patients with proliferative diabetic retinopathy, suggesting its possible role in diabetic retinopathy (Ma et al., 1996). The results of the present invention demonstrated an anti-angiogenic activity of kallistatin in a retinal neovascularization model. Moreover, the present invention has also revealed another new activity of this serpin, i.e., decreasing vascular permeability and vascular leakage.

As kallistatin can inhibit the releases of bioactive kinins from kininogen (Zhou et al., 1992), and kinin promotes angiogenesis through the B1 receptor (Hu et al., 1993; Emanueli et al., 2002), a natural question is whether the anti-angiogenic activity of kallistatin is through its inhibition of kallikrein activity and consequent reduction of kinin production. The present invention has employed selective B1 and B2 kinin receptor antagonists to treat endothelial cells and compare their inhibitory effects with that of kallistatin alone. It has been shown previously that at 1 mM, the B1 receptor antagonist des-Arg9-[Leu8]-bradykinin is able to completely block bradykinin-induced endothelial cell proliferation (Morbidelli et al., 1998). Here, a high concentration (5 mM) of des-Arg9-[Leu8]-bradykinin was used to ensure a complete blockade of the B1 receptor. The results showed that the inhibitory effect of RCEC by complete blockade of the B1 receptor was significantly weaker than that of kallistatin alone (P<0.01), while blocking the B2 receptor had no inhibition. These results demonstrate that the anti-angiogenic activity of kallistatin cannot be ascribed to the inhibition of kinin production. This observation is consistent with previous findings by Chao's group (Chao et al., 2001; Miao et al., 2002). It is possible that kallistatin is a multi-functional protein which has several independent activities, i.e., binding with tissue kallikrein, inhibiting angiogenesis and decreasing vascular permeability. It is proposed that these functions involve distinct structural domains in kallistatin. The multi-functional feature has also been documented in other serpins. Antithrombin III is known to inhibit thrombin and also has anti-angiogenic activity (O'Reilly et al., 1999). PEDF, a non-inhibitory serpin, possesses both neurotrophic and anti-angiogenic activities (Dawson et al., 1999; Becerra et al., 1995).

In the past few years, a number of endogenous angiogenic inhibitors have been identified. Most of these inhibitors can be classified into two major groups: serpins including PEDF, maspin and anti-thrombin III (Dawson et al., 1999; O'Reilly et al., 1999; Zhang et al., 2000), and peptide fragments of extracellular proteins including endostatin, angiostatin, K5 and tumstatin (O'Reilly et al., 1997; O'Reilly et al., 1994; Cao et al., 1997; Cao et al., 1996; Maeshima et al., 2002). Recently, it has been shown that several fragments of extracellular proteins, e.g., angiostatin, endostatin and tumstatin bind to integrins, and their anti-angiogenic activities have been suggested to be through interfering with integrin signaling (Maeshima et al., 2002; Tarui et al., 2001; Rehn et al., 2001). However, the molecular mechanisms of the anti-angiogenic serpins are still unknown. The results described herein demonstrate that kallistatin inhibits VEGF binding to its receptors on endothelial cells. Efficient binding of VEGF to its receptors is known to depend for heparin binding (Tessler et al., 1994; Gitay-Goren et al., 1992). As kallistatin is also a heparin-binding protein (Chao et al., 1990), the inhibition of VEGF binding to its receptors by kallistatin may be through competing on heparin binding. The results described herein also demonstrate that kallistatin down-regulates VEGF expression under hypoxia. The mechanism responsible for kallistatin-mediated down-regulation of VEGF is presently unknown. VEGF is a potent endothelial cell growth factor, and elevated VEGF levels are a major cause of pathological angiogenesis and vascular leakage as found in diabetic retinopathy (Robinson et al., 1998). Inhibition of VEGF binding to its receptors and down-regulation of endogenous VEGF may represent a mechanism underlying the anti-angiogenic activity of kallistatin and its effect on vascular leakage.

Anti-angiogenic proteins or peptide fragments can offset increased angiogenic stimulators under hypoxia, and thus are believed to have therapeutic potential. Moreover, reduction of vascular leakage by kallistatin can be a beneficial effect in the treatment of macular edema in diabetic retinopathy. Kallistatin can be produced with a high yield in E. coli as a soluble protein with kallikrein-binding activity and inhibitory effects on angiogenesis and vascular leakage (Ma et al., 1993). It is relatively stable and has low cytotoxicity to other cell types including pericytes and Müller cells. Intravitreal injection of kallistatin does not cause any detectable inflammatory response or toxicity to retinal tissues and normal vasculature. Moreover, kallistatin is endogenously expressed in multiple tissues including the retina and vitreous. These features suggest that kallistatin is a promising candidate for effective anti-angiogenic reagents in the treatment of neovascular disorders and vascular leakage such as proliferative diabetic retinopathy and solid tumors.

### EXAMPLE 2

### Therapeutic Potential of Kallistatin in Diabetic Nephropathy (DN), Inflammation and Fibrosis

Kallistatin has displayed beneficial effects on retinal neovascularization and vascular leakage, as it inhibits VEGF over-expression in diabetic retinopathy model and blocks VEGF binding to VEGF receptors. Kallistatin levels are decreased in the vitreous and retina of diabetic animal model and diabetic patients. To determine if kallistatin is implicated in diabetic kidney complications, kallistatin levels were measured in the kidney.

Diabetes was induced in Brown Norway rats by an injection of streptozotocin (STZ). Glucose levels were measured at 48 h after the STZ injection. Only rats with glucose levels higher than 350 mg/dl were considered diabetic. The glucose levels were monitored every week thereafter. Six weeks after the STZ injection (at this time point, several abnormalities in the renal functions such albuminuria and polyuria had occurred), 5 of the diabetic rats and five of age-matched normal controls were euthanized. The kidneys were dissected and homogenized. Protein concentrations in the soluble fraction were measured by BioRad protein assay. Kallistatin levels were measured by a specific ELISA and normalized by total soluble proteins.

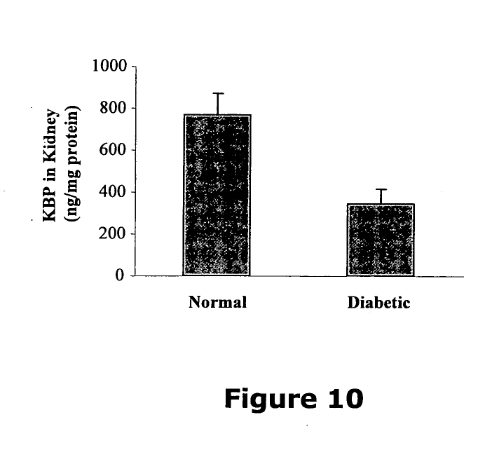

The results showed that diabetic kidneys have significantly lower kallistatin levels than that in normal controls (P<0.01) (FIG. 10). This demonstrates that decreased kallistatin could contribute to the development of DN.

Kallistatin blocks high glucose concentration-induced fibrosis in kidney cells. Hyperglycemia is known to induce a series of kidney changes in DN, including fibrosis and inflammation. High glucose induced fibronectin over-production from renal mesangial cells is a major step in kidney fibrosis and mesangial expansion in DN.

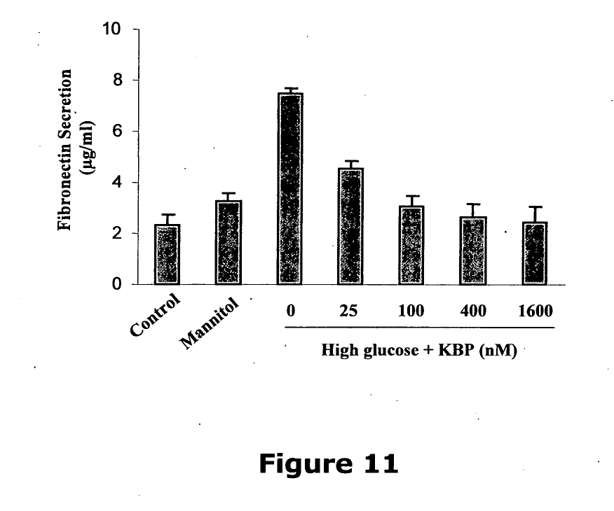

Cultured primary human mesangial cells (HMC) were treated with 30 mM glucose in the absence or presence of different concentrations of kallistatin (25-1600 nM) for 3 days. Cells cultured in 5 mM glucose were used as a control. To exclude the possible effect of osmolarity from high glucose, an osmolarity control was also included which was treated with 5 mM glucose and 25 mM mannitol. After the treatments for 3 days, secretion of fibronectin into the culture medium was measured. The results showed that high glucose induced over-production of fibronectin from HMC. Kallistatin showed a concentration-dependent decrease of fibronectin production with doses of 25 to 1600 nM (FIG. 11). These results demonstrate that kallistatin has an anti-fibrosis activity, and thus has therapeutic potential in diseases with fibrosis such as DN and chronic inflammation.

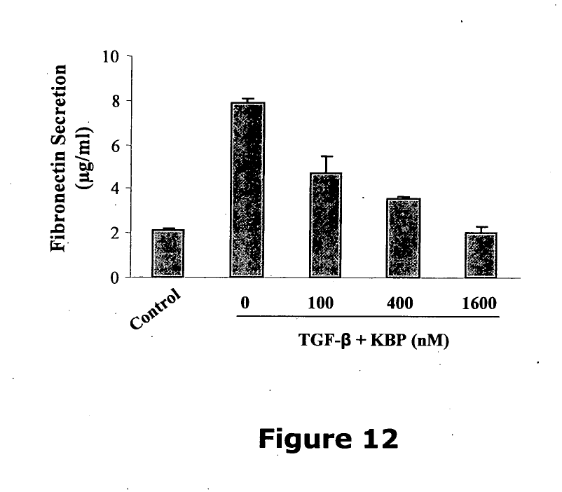

Kallistatin blocks the function of TGF-β, a major pathogenic factor in DN. TGF-β is a major inflammatory and fibrosis mediator. It plays a major role in the development of DN. To explore the role of kallistatin in DN, the effect of kallistatin in blocking TGF-β activity in kidney cells was determined. HMC were treated with 5 ng/ml TGF-β for 3 days without or with different concentrations of kallistatin. The results showed that TGF-β significantly induced fibronectin over-secretion in the medium, while kallistatin blocked the TGF-β-induced fibronectin secretion in a concentration-dependent manner (FIG. 12). This finding demonstrates that kallistatin functions as an endogenous antagonist of TGF-β, and thus has a protective effect against fibrosis and inflammation induced by TGF-β in diabetic kidney.

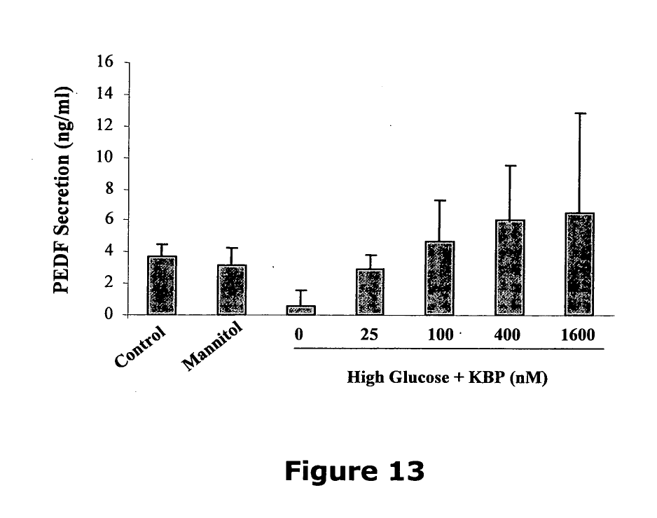

Kallistatin up-regulates endogenous anti-inflammatory factors in the kidney. Pigment epithelium-derived factor (PEDF) is an anti-angiogenic factor. Recently, the inventor has shown that PEDF also has anti-inflammatory activities and has a protective effect against DN (see U.S. Ser. No. 10/963,115, filed Oct. 12, 2004, the contents of which are hereby expressly incorporated herein by reference). Decreased PEDF levels in diabetic kidney may contribute to the development of DN. The effect of kallistatin on PEDF expression has been determined in kidney cells. HMC were treated with high glucose (30 mM glucose) with different concentrations of kallistatin for 3 days. The PEDF levels in the cultured medium were measured by ELISA specific for PEDF. As shown in FIG. 13, high glucose significantly decreased PEDF levels, consistent with the in vivo finding in diabetic kidney. Kallistatin reversed the changes of PEDF under high glucose conditions, suggesting that kallistatin rescues the endogenous anti-inflammatory factors, and thus has anti-inflammation activities.

Taken together, these data demonstrate that kallistatin is an anti-fibrosis and anti-inflammatory factor in the kidney. These activities may be via inhibiting TGF-β and VEGF, two major inflammatory factors in the kidney. The decreased kallistatin levels in diabetic kidney may be responsible for the pathogenesis of DN. Therefore, kallistatin should have a beneficial effect in the treatment of DN and other inflammatory and fibrosis diseases.

### MATERIALS AND METHODS

Materials: The rat Müller cell line, rMC-1, was a generous gift from Dr. Vjay Sarthy at the Northwestern University. Retinal capillary endothelial cells (RCEC) and pericytes were isolated from bovine eyes following a protocol described previously (Grant et al., 1991; Gitlin et al., 1983). The identity of RCEC was confirmed by a characteristic cobblestone morphology and the incorporation of acetylated low-density lipoprotein labeled with a fluorescent probe, DiI (1,1′-dioctadecyl-3,3,3′,3′-tetramethylindocarbocyanine perchlorate) (Biomedical Technologies Inc., Stoughton, Mass.). Purity of the pericyte culture was determined by immunostaining using an FITC-conjugated antibody specific to a-smooth-muscle actin (Sigma, St. Louis, Mo.).

Brown Norway rats were purchased from Harlan (Indianapolis, Ind.). Care, use and treatment of all animals in this study were in strict agreement with the ARVO Statement for the Use of Animals in Ophthalmic and Vision Research, as well as the guidelines set forth in the Care and Use of Laboratory Animals by the Medical University of South Carolina.

Expression and purification of recombinant kallistatin: The kallistatin cDNA containing a sequence coding for the full-length mature peptide was amplified from the total RNA of rat liver by reverse transcription-polymerase chain reaction (RT-PCR) as described previously (Ma et al., 1995). The 5′ PCR primer (5′-GTCGGATCCTGATGGCATACTGGGAAG-3′) (SEQ ID NO:3) and the 3′ primer (5′-GTGGAGCTCATGGGGTTAGTGACTTTG-3′) (SEQ ID NO:4) contain a BamHI and SacI site, respectively. The PCR product was cloned into the pET28 vector (Novagen, Inc., Madison, Wis.) at the BamHI and SacI sites in frame with the sequence encoding the 6×His tag at its 3′ end.

The kallistatin/pET28 construct was introduced into E. coli strain BL-21/DE3 (Novagen, Inc., Madison, Wis.). The expression and purification were performed as described previously (Zhang et al., 2001). Endotoxin levels were monitored using a limulus amebocyte kit (Biowhittaker, Walkersville, Md.).

Quantification of viable cells: Cells were plated in 12-well plates in triplicate and cultured in the growth medium until they reached 60-70% of confluency. The culture medium was replaced with a medium containing 1% fetal bovine serum (FBS). Recombinant kallistatin was added to the cover medium to various concentrations and incubated with the cells for 72 h. The viable cells were quantified by the MTT (3-[4,5-dimethylthiazol-2-yl]-2,5-dephenyl tetrazolium bromid, Roche, Mannheim, Germany) assay following a protocol recommended by the manufacturer. The effect of kallistatin on viable cell number was analyzed using Student's t test.

[³H] thymidine incorporation assay: RCEC were seeded in 24-well plates in 1:1 DMEM+F-12 nutrient mixture plus 10% FBS and cultured in a CO2 incubator to reach 60-70% confluency. The cells were washed 3 times with PBS and the growth medium replaced by a medium containing 1% FBS and different concentrations of kallistatin. After 24 h culture, [³H] thymidine was added to the medium (2 μCi/well) and incubated with the cells at 37° C. for 12 h. Free [³H] thymidine was removed by 3 washes with PBS, and a solution of 6% TCA was added to the wells. The TCA solution was removed, and the wells were washed once with PBS. The remaining material was solubilized with 200 μl of 1 M NaOH (Smith et al., 1999). Incorporated [³H] thymidine was measured with a microplate scintillation counter (Packard Instrument Company, Meriden, Conn.).

Quantitative analysis of apoptosis by flow cytometry: RCEC were plated at a density of 105 cells/well in 6-well plates. Two days after seeding, the cells were exposed to kallistatin at a different concentration for 24 h and harvested for Annexin and propidium iodide (PI) staining using the Annexin V-FITC Apoptosis Detection Kit (Sigma, St. Louis, Mich.) following the protocol recommended by the manufacturer. Colchicine (Sigma, St. Louis, Mich.) which is known to induce apoptosis by disrupting microtubules and preventing its polymerization was used as a positive control. The cells were subsequently counted by flow cytometry.

Induction of retinal neovascularization and intravitreal injection of kallistatin: Retinal neovascularization was induced as described by Smith et al. (Smith et al., 1994) with some modifications. Briefly, newborn pigmented Brown Norway rats at postnatal day 7 (P7) were exposed to hyperoxia (75% O₂) for 5 days and then normoxia. Animals were anesthetized, and kallistatin was injected into the vitreous of the right eye through the pars plana using a glass capillary. The left eye received the same volume of PBS as the control. After injection, the animals were kept in normoxia for another 5 days for further analyses.

Retinal angiography with high molecular weight fluorescein and quantification of neovascularization: Retinal angiography was as described by Smith et al. (Smith et al., 1994). Briefly, rats were anesthetized and perfused with fluorescein via intra-ventricle injection of 50 mg/ml of high molecular weight (2×10⁶) fluorescein isothiocyanate-dextran (Sigma, St. Louis, Mo.). The animals were immediately sacrificed, and the eyes were enucleated and fixed in 4% paraformaldehyde for 10 min. The retina was dissected free of the lens and vitreous and incubated in 4% paraformaldehyde for 3 h. The retina was cut and flat-mounted on a gelatin-coated slide. The vasculature was then examined under a fluorescent microscope (Axioplan2 Imaging, Zeiss).

Retinal neovascularization was quantified by counting pre-retinal vascular cells as previously described (Zhang et al., 2001). The average number of pre-retinal vascular nuclei was compared to the PBS control group by Student's t test.

Measurement of vascular permeability: Vascular permeability was quantified by measuring albumin leakage from blood vessels into the retina, iris and choroid using Evans blue following a documented protocol (Xu et al., 2001) with minor modifications. Evans blue dye (Sigma, St. Louis, Mo.) was dissolved in normal saline (30 mg/ml), sonicated for 5 min and filtered through a 0.45-μm filter (Millipore, Bedford, Mass.). The rats were anesthetized, and Evans blue (30 mg/kg) was injected over 10 seconds through the femoral vein using a glass capillary under microscopic inspection. Evans blue non-covalently binds to plasma albumin in the blood stream (Radius et al., 1980). Immediately after Evans blue infusion, the rats turned visibly blue, confirming their uptake and distribution of the dye. The rats were kept on a warm pad for 2 h to ensure the complete circulation of the dye. Then the chest cavity was opened, and the rats were perfused via the left ventricle with 1% paraformaldehyde in citrate buffer (pH=4.2) which was pre-warmed to 37° C. to prevent vasoconstriction. The perfusion lasted 2 min under the physiological pressure of 120 mmHg to clear the dye from the vessel. Immediately after perfusion, the eyes were enucleated and the retina, iris and choroid were carefully dissected under an operating microscope. Evans blue dye was extracted by incubating each sample in 150 μl formamide for 18 h at 70° C. The extract was centrifuged (TL; Beckman) at 70,000 rpm (Rotor type: TLA 100.3) for 20 min at 4° C. Absorbance was measured using 100 μl of the supernatant at 620 nm. The concentration of Evans blue in the extracts was calculated from a standard curve of Evans blue in formamide and normalized by the total protein concentration in each sample. Results were expressed in micrograms of Evans blue per milligrams of total protein content.

VEGF binding assay: VEGF (PeproTech, Inc., Rocky Hill, N.J.) was labeled with 125I using the Chloromine T ¹²⁵I Labeling Kit (ICN Pharmaceuticals, Inc. Costa Mesa, Calif.) following a protocol recommended by the manufacturer. For the binding assay, RCEC were seeded in 12-well plates and cultured until 80% confluency was reached. The culture medium was replaced with serum-free medium. ¹²⁵I-VEGF was added to the medium, 2.5×10⁵ CPM/well with and without different concentrations of kallistatin or recombinant plasminogen kringle 5 (K5) and incubated with the cells for 1 h. The medium was removed and cells washed three times with PBS. The cells were then lysed by the addition of 0.35 ml 10% SDS. The cell lysates were collected, and the 125I-VEGF bound to RCEC was quantified by a gamma counter.

Measurement of VEGF in the conditioned medium of RCEC by ELISA: RCEC were seeded in T75 flasks in endothelial cell growth medium and cultured in a CO₂ incubator to reach 60-70% confluency. The cells were washed 3 times with PBS and the growth medium replaced by a serum-free medium containing bFGF (GIBCO-BRL, Gaithersburg, Md.). Kallistatin was added to the medium to various concentrations and incubated with the cells for 24 h under normoxia or hypoxia (in a chamber that was perfused with a mixture of 95% N₂+5% CO₂). The conditioned medium was harvested for VEGF ELISA and the cells were used for Western blot analysis. The conditioned medium was centrifuged and the protein concentration in the supernatant was measured with BioRad protein assay. VEGF concentration was measured using a VEGF ELISA kit (R& D systems, Minneapolis, Minn.) and normalized by total protein concentration in the medium.

Western blot analysis: One hundred micrograms of total protein were used for Western blot analysis of VEGF using an ECL detection kit (Amersham International plc, Piscataway, N.J.) (Gao et al., 2001). The same membrane was stripped and re-blotted with an antibody specific to β-actin. VEGF levels were normalized by β-actin.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents and peptides which are both chemically and physiologically related may be substituted for the agents and peptides described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Aiello L P (1997). Vascular endothelial growth factor and the eye: biochemical mechanisms of action and implications for novel therapies. Ophthalmic Res 29: 354-362.
2. Aiello, L P, Bursell, S E, Clermont, A, Duh, E, Ishii, H, Takagi, C, Mori, F, Ciulla, T A, Ways, K, Jirousek, M, et al. (1997). Vascular endothelial growth factor-induced retinal permeability is mediated by protein kinase C in vivo and suppressed by an orally effective beta-isoform-selective inhibitor. Diabetes 46:1473-1480.
3. Aiello, L P, and Wong, J S (2000). Role of vascular endothelial growth factor in diabetic vascular complications. Kidney Intl.—Suppl. 77:S113-119.
4. American Diabetes Association (2000). Diabetic nephropathy. Diabetes Care. 23: S69-72.
5. Anonymous (1995). Effect of intensive therapy on the development and progression of diabetic nephropathy in the Diabetes Control and Complications Trial. The Diabetes Control and Complications (DCCT) Research Group. Kidney Int 47: 1703-1720.
6. Anonymous (2000). Retinopathy and nephropathy in patients with type 1 diabetes four years after a trial of intensive therapy. The Diabetes Control and Complications Trial/Epidemiology of Diabetes Interventions and Complications Research Group.[erratum appears in N Engl J Med 2000 May 4;342(18):1376]. New Engl J Med 342: 381-389.
7. Antonetti, D A, Lieth, E, Barber, A J, and Gardner, T W (1999). Molecular mechanisms of vascular permeability in diabetic retinopathy. Sem. Ophthalmol. 14:240-248.
8. Becerra S P, Sagasti A, Spinella P, Notario V (1995). Pigment epithelium-derived factor behaves like a noninhibitory serpin. Neurotrophic activity does not require the serpin reactive loop. J Biol Chem 270: 25992-25999.
9. Bhoola K D, Figueroa C D, Worthy K (1992). Bioregulation of kinins: kallikreins, kininogens, and kininases. Pharmacological Reviews 44: 1-80.
10. Blom M L, Green W R, Schachat A P (1994). Diabetic retinopathy: a review. Del Med J 66: 379-388.
11. Bolton, S J, Anthony, D C, and Perry, V H (1998). Loss of the tight junction proteins occludin and zonula occludens-1 from cerebral vascular endothelium during neutrophil-induced blood-brain barrier breakdown in vivo. Neuroscience. 86:1245-1257.
12. Bortoloso, E, Del Prete, D, Gambaro, G, Dalla Vestra, M, Sailer, A, Baggio, B, Antonucci, F, Fioretto, P, and Anglani, F (2001). Vascular endothelial growth factor (VEGF) and VEGF receptors in diabetic nephropathy: expression studies in biopsies of type 2 diabetic patients. Renal Failure. 23:483-493.
13. Bussolino F, Mantovani A, Persico G (1997). Molecular mechanisms of blood vessel formation. Trends Biochem Sci 22: 251-256.
14. Cao Y, Chen A, An SSA (1997). Kringle 5 of plasminogen is a novel inhibitor of endothelial cell growth. J Biol Chem 272: 22924-22928.
15. Cao Y, Ji R W, Davidson D (1996). Kringle domains of human angiostatin. Characterization of the anti-proliferative activity on endothelial cells. J Biol Chem 271: 29461-29467.
16. Chai K X, Ma J-X, Murray S R, Chao J, Chao L (1991). Molecular cloning and analysis of the rat kallikrein-binding protein gene. J Biol Chem 266: 16029-16036.
17. Chao J, Tillman D M, Wang M Y, Margolius H S, Chao L (1986). Identification of a new tissue-kallikrein-binding protein. Biochem J 239: 325-331.
18. Chao J, Chai K X, Chen L M et al. (1990). Tissue kallikrein-binding protein is a serpin. I. Purification, characterization, and distribution in normotensive and spontaneously hypertensive rats. J Biol Chem 265: 16394-16401.
19. Chao J, Miao R Q, Chen V, Chen L M, Chao L (2001). Novel roles of kallistatin, a specific tissue kallikrein inhibitor, in vascular remodeling. Biol Chem 382: 15-21.
20. Chen L M, Ma J-X, Liang Y M, Chao L, Chao J (1996). Tissue kallikrein-binding protein reduces blood pressure in transgenic mice. J Biol Chem 271: 27590-27594.
21. Chiarelli F, Santilli F, Mohn A (2000). Role of growth factors in the development of diabetic complications. Horm Res 53: 53-67.
22. Cooper M E (2001). Interaction of metabolic and haemodynamic factors in mediating experimental diabetic nephropathy. Diabetologia. 44: 1957-1972.
23. Cunha-Vaz, J G, Gray, J R, Zeimer, R C, Mota, M C, Ishimoto, B M, and Leite, E (1985). Characterization of the early stages of diabetic retinopathy by vitreous fluorophotometry. Diabetes 34:53-59.
24. Dawson D W, Volpert O V, Gillis P, et al. (1999). Pigment epithelium-derived factor: a potent inhibitor of angiogenesis. Science 285: 245-248.
25. Del Maschio, A, Zanetti, A, Corada, M, Rival, Y, Ruco, L, Lampugnani, M G, and Dejana, E (1996). Polymorphonuclear leukocyte adhesion triggers the disorganization of endothelial cell-to-cell adherens junctions. J. Cell Biol. 135:497-510.
26. Dvorak H F, Brown L F, Detmar M, Dvorak A M (1995). Vascular permeability factor/vascular endothelial growth factor, microvascular hyperpermeability, and angiogenesis. Am J Pathol 146: 1029-1039.
27. Ellis D, Forrest K Y, Erbey J, Orchard T J (1998). Urinary measurement of transforming growth factor-beta and type IV collagen as new markers of renal injury: application in diabetic nephropathy. Clin Chem 44: 950-956.
28. Emanueli C, Bonaria Salis M, Stacca T et al. (2002). Targeting kinin B(1) receptor for therapeutic neovascularization. Circulation 105: 360-366.
29. Gambaro G, Ceol M, Del Prete D, D'Angelo A (2000). GLUT-1 and TGF-beta: the link between hyperglycaemia and diabetic nephropathy.[comment]. Nephrol Dial Transplant 15: 1476-1477.
30. Gao G Q, Li Y, Zhang D C, Gee S, Crosson C, Ma J-X (2001). Unbalanced expression of VEGF and PEDF in ischemia-induced retinal neovascularization. FEBS Lett 489: 270-276.
31. Gitay-Goren H, Soker S, Vlodavsky I, Neufeld G (1992). The binding of vascular endothelial growth factor to its receptors is dependent on cell surface-associated heparin-like molecules. J Biol Chem 267: 6093-6098.
32. Gitlin J D, D'Amore P A (1983). Culture of retinal capillary cells using selective growth media. Microvascular Research 26: 74-80.
33. Goldfarb, S, and Ziyadeh, F N (2001). TGF-beta: a crucial component of the pathogenesis of diabetic nephropathy. Transactions of the American Clinical & Climatological Association. 112:27-32; discussion 33.
34. Grant M B, Guay C (1991). Plasminogen activator production by human retinal endothelial cells of nondiabetic and diabetic origin. Invest Ophthalmol Vis Sci 32: 53-64.
35. Greener, M (2000). Targeting TGF could counter diabetic nephropathy. Molecular Medicine Today. 6:376.
36. Hammes, H P, Lin, J, Bretzel, R G, Brownlee, M, and Breier, G (1998). Upregulation of the vascular endothelial growth factor/vascular endothelial growth factor receptor system in experimental background diabetic retinopathy of the rat. Diabetes. 47:401-406.
37. Hatcher H C, Ma J-X, Chao J, Chao L, Ottlecz A (1997). Kallikrein-binding protein levels are reduced in the retinas of streptozotocin-induced diabetic rats. Invest Ophthalmol Vis Sci 38: 658-664.
38. Hatcher H C, Wright N M, Chao J, Chao L, Ma J-X (1999). Kallikrein-binding protein is induced by growth hormone in the dwarf rat. FASEB J 13: 1839-1844.
39. Hu D E, Fan T P (1993). [Leu8]des-Arg9-bradykinin inhibits the angiogenic effect of bradykinin and interleukin-1 in rats. Br J Pharmacol 109: 14-17.
40. Iglesias-de la Cruz M C, Ziyadeh F N, Isono M, Kouahou M, Han D C, Kalluri R, Mundel P, Chen S (2002). Effects of high glucose and TGF-beta1 on the expression of collagen IV and vascular endothelial growth factor in mouse podocytes. Kidney Int. 62: 901-913.
41. Jacobson B, Basu P K, Hasany S M (1984). Vascular endothelial cell growth inhibitor of normal and pathologic human vitreous. Arch Ophthalmol 102: 1543-1545.
42. Jimenez B, Volpert O V (2001). Mechanistic insights on the inhibition of tumor angiogenesis. J Mol Med 78: 663-672.
43. Kim B S, Goligorsky M S (2003). Role of VEGF in kidney development, microvascular maintenance and pathophysiology of renal disease. Korean J Intern Med 18: 65-75.
44. Lane P H, Snelling D M, Langer W J (2001). Streptozocin diabetes elevates all isoforms of TGF-beta in the rat kidney. Int J Exp Diabetes Res 2: 55-62.
45. Lutty G A, Thompson D C, Gallup J Y, Mello R J, Patz A, Fenselau A (1983). Vitreous: an inhibitor of retinal extract-induced neovascularization. Invest Ophthalmol Vis Sci 24: 52-56.
46. Lutty G A, Mello R J, Chandler C, Fait C, Bennett A, Patz A (1985). Regulation of cell growth by vitreous humour. J Cell Sci 76: 53-65.
47. Ma J-X, Chao L, Zhou G, Chao J (1993). Expression and characterization of rat kallikrein-binding protein in Escherichia coli. Biochem J 292: 825-832.
48. Ma J-X, King L, Yang Z, Crouch R K, Chao L, Chao J (1996). Quantitative comparison of kallistatin in non-diabetic and diabetic vitreous fluids. Curr Eye Res 15: 1117-1123.
49. Ma J-X, Yang Z, Chao J, Chao L (1995). Intramuscular delivery of rat kallikrein-binding protein gene reverses hypotension in transgenic mice expressing human tissue kallikrein. J Biol Chem 270: 451-455.
50. Ma J-X, Zhang D, Laser M et al. (1999). Identification of RPE65 in transformed kidney cells. FEBS Lett 452: 199-204.
51. Maeshima Y, Sudhakar A, Lively J C (2002). Tumstatin, an endothelial cell-specific inhibitor of protein synthesis. Science 295: 140-143.
52. Miao R Q, Agata J, Chao L, Chao J (2002). Kallistatin is a new inhibitor of angiogenesis and tumor growth. Blood 100: 3245-3252.
53. Miller J W (1997). Vascular endothelial growth factor and ocular neovascularization. Am J Pathol 151: 13-23.
54. Miyamoto, K, Khosrof, S, Bursell, S E, Moromizato, Y, Aiello, L P, Ogura, Y, and Adamis, A P (2000). Vascular endothelial growth factor (VEGF)—induced retinal vascular permeability is mediated by intercellular adhesion molecule-1 (ICAM-1). Am. J. Pathol. 156:1733-1739.
55. Mogyorosi A, Kapoor A, Isono M, Kapoor S, Sharma K, Ziyadeh F N (2000). Utility of serum and urinary transforming growth factor-beta levels as markers of diabetic nephropathy. Nephron 86: 234-235.
56. Morbidelli L, Parenti A, Giovannelli L, Granger H J, Ledda F, Ziche M (1998). B1 receptor involvement in the effect of bradykinin on venular endothelial cell proliferation and potentiation of FGF-2 effects. Br J Pharmacol 124: 1286-1292.
57. Moss, S E, Klein, R, and Klein, B E (1998). The 14-year incidence of visual loss in a diabetic population. Ophthalmology. 105:998-1003.
58. Murata, T, Nakagawa, K, Khalil, A, Ishibashi, T, Inomata, H, and Sueishi, K (1996). The relation between expression of vascular endothelial growth factor and breakdown of the blood-retinal barrier in diabetic rat retinas. Lab. Invest. 74:819-825.
59. Ng D P, Warram J H, Krolewski A S (2003). TGF-beta 1 as a genetic susceptibility locus for advanced diabetic nephropathy in type 1 diabetes mellitus: an investigation of multiple known DNA sequence variants. Am J Kidney Dis 41: 22-28.
60. O'Reilly M S, Pirie-Shepherd S, Lane W S, Folkman J (1999). Antiangiogenic activity of the cleaved conformation of the serpin antithrombin. Science 285: 1926-1928.
61. O'Reilly M S, Boehm T, Shing Y (1997). Endostatin: an endogenous inhibitor of angiogenesis and tumor growth. Cell 88: 277-285.
62. O'Reilly M S, Holmgren L, Shing Y (1994). Angiostatin: a novel angiogenesis inhibitor that mediates the suppression of metastases by a Lewis lung carcinoma. Cell 79: 315-328.
63. Pfeiffer A, Middelberg-Bisping K, Drewes C, Schatz H (1996). Elevated plasma levels of transforming growth factor-beta 1 in NIDDM. Diabetes Care 19: 1113-1117.
64. Pierce E A, Avery R L, Foley E D, Aiello L P, Smith L E (1995). Vascular endothelial growth factor/vascular permeability factor expression in a mouse model of retinal neovascularization. Proc Natl Acad Sci USA 92: 905-909.
65. Preis I, Langer R, Brem H, Folkman J (1977). Inhibition of neovascularization by an extract derived from vitreous. Am J Ophthalmol 84: 323-328.
66. Qaum, T, Xu, Q, Joussen, A M, Clemens, M W, Qin, W, Miyamoto, K, Hassessian, H, Wiegand, S J, Rudge, J, Yancopoulos, G D, et al. (2001). VEGF—initiated blood-retinal barrier breakdown in early diabetes. Invest. Ophthalmol. Vis. Sci. 42:2408-2413.
67. Radius R L, Anderson D R (1980). Distribution of albumin in the normal monkey eye as revealed by Evans blue fluorescence microscopy. Invest Ophthalmol Vis Sci 19: 238-243.
68. Raptis, A E, and Viberti, G (2001). Pathogenesis of diabetic nephropathy. Experimental & Clinical Endocrinology & Diabetes. 109:S424-437.
69. Raymond L, Jacobson B (1982). Isolation and identification of stimulatory and inhibitory cell growth factors in bovine vitreous. Exp Eye Res 34: 267-286.
70. Rehn M, Veikkola T, Kukk-Valdre E et al. (2001). Interaction of endostatin with integrins implicated in angiogenesis. Proc Natl Acad Sci USA 98: 1024-1029.
71. Robinson G S, Aiello L P (1998). Angiogenic factors in diabetic ocular disease: mechanisms of today, therapies for tomorrow. Int Ophthalmol Clin 38: 89-102.
72. Sakharova, O V, Taal, M W, and Brenner, B M (2001). Pathogenesis of diabetic nephropathy: focus on transforming growth factor-beta and connective tissue growth factor. Current Opinion in Nephrology & Hypertension. 10:727-738.
73. Schachter M (1983). Kallikreins and kinins, an overview: some thoughts old and new. Adv Exp Med Biol 156: 13-27.
74. Senthil D, Choudhury G G, McLaurin C, Kasinath B S (2003). Vascular endothelial growth factor induces protein synthesis in renal epithelial cells: a potential role in diabetic nephropathy.[see comment]. Kidney Int 64: 468-479.
75. Silverman G A, Bird P I, Carrell R W et al. (2001). The serpins are an expanding superfamily of structurally similar but functionally diverse proteins. Evolution, mechanism of inhibition, novel functions, and a revised nomenclature. J Biol Chem 276: 33293-33296.
76. Smith L E, Shen W, Perruzzi C et al. (1999). Regulation of vascular endothelial growth factor-dependent retinal neovascularization by insulin-like growth factor-1 receptor. Nature Med 5: 1390-1395.
77. Smith L E, Wesolowski E, McLellan A (1994). Oxygen-induced retinopathy in the mouse. Invest Ophthalmol Vis Sci 35: 101-111.
78. Spranger J, Hammes H P, Preissner K T, Schatz H, Pfeiffer A F (2000). Release of the angiogenesis inhibitor angiostatin in patients with proliferative diabetic retinopathy: association with retinal photocoagulation. Diabetologia 43: 1404-1407.
79. Spranger J, Osterhoff M, Reimann M et al. (2001). Loss of the antiangiogenic pigment epithelium-derived factor in patients with angiogenic eye disease. Diabetes 50: 2641-2645.
80. Tamaki, K, and Okuda, S (2003). Role of TGF-beta in the progression of renal fibrosis. Contributions to Nephrology. 139:44-65.
81. Tarui T, Miles L A, Takada Y (2001). Specific interaction of angiostatin with integrin alpha(v)beta(3) in endothelial cells. J Biol Chem 276: 39562-39568.
82. Tessler S, Rockwell P, Hicklin D (1994). Heparin modulates the interaction of VEGF165 with soluble and cell associated flk-1 receptors. J Biol Chem 269: 12456-12461.
83. Xu Q, Qaum T, Adamis A P (2001). Sensitive blood-retinal barrier breakdown quantitation using Evans blue. Invest Ophthalmol Vis Sci 42: 789-794.
84. Yoon J B, Towle H C, Seelig S (1987). Growth hormone induces two mRNA species of the serine protease inhibitor gene family in rat liver. J Biol Chem 262: 4284-4289.
85. Yoshida, A, Ishiko, S, Kojima, M, and Ogasawara, H (1993). Permeability of the blood-ocular barrier in adolescent and adult diabetic patients. Br. J. Ophthalmol. 77:158-161.
86. Zhang D, Kaufman P L, Gao G, Saunders R A, Ma J-X (2001). Intravitreal injection of plasminogen kringle 5, an endogenous angiogenic inhibitor, arrests retinal neovascularization in rats. Diabetologia 44: 757-765.
87. Zhang M, Volpert O, Shi Y H, Bouck N (2000). Maspin is an angiogenesis inhibitor. Nature Med 6: 196-199.
88. Zhang, S X, Sima, J, Shao, C, Fant, J, Chen, Y, Rohrer, B, Gao, G and Ma, J-X (2004). Diabetologia 47, 124-131.
89. Zhou G X, Chao L, Chao J (1992). Kallistatin: a novel human tissue kallikrein inhibitor. Purification, characterization, and reactive center sequence. J Biol Chem 267: 25873-25880.

What is claimed is:

1. A method of inhibiting vascular leakage in an animal who is not exhibiting pathological angiogenesis, comprising the step of:
administering to an animal in need thereof an effective amount of a composition capable of inhibiting vascular leakage, wherein the composition comprises kallistatin, and wherein the effective amount of the composition is insufficient to inhibit pathological angiogenesis.

2. The method of claim 1 wherein the animal has a disease selected from the group consisting of diabetes, chronic inflammation, brain edema, arthritis, uvietis, macular edema, hyperglycemia, a kidney inflammatory disease, a disorder resulting in kidney fibrosis, a disorder of the kidney resulting in proteinuria, and combinations thereof.

3. The method of claim 2, wherein the animal has diabetic nephropathy, and the vascular leakage is associated with the diabetic nephropathy.

4. The method of claim 2, wherein the animal has diabetic retinopathy, and the vascular leakage is associated with the diabetic retinopathy.

5. The method of claim 1 wherein the effective amount of the composition causes a statistically significant inhibition of binding of Vascular Endothelial Growth Factor (VEGF) to VEGF receptors.

6. The method of claim 1 wherein the composition is a natural peptide that exhibits substantially no toxicity in the animal.

7. The method of claim 1 wherein the animal is a mammal.

8. The method of claim 1 wherein the animal is a human.

9. The method of claim 1 wherein the effective amount of the composition causes a statistically significant inhibition of endogenous VEGF expression.

10. The method of claim 1 wherein the effective amount of the composition causes a statistically significant inhibition of endogenous TGF-β expression.

11. The method of claim 1 wherein the composition is recombinantly-produced kallistatin.

12. A method of inhibiting vascular leakage prior to onset of pathological angiogenesis, the method comprising the step of:
administering to an animal in need thereof an effective amount of a composition capable of inhibiting vascular leakage prior to onset of pathological angiogenesis, wherein the composition comprises recombinantly-produced kallistatin, and wherein the effective amount of the composition is insufficient to inhibit pathological angiogenesis.

13. The method of claim 12 wherein the animal has a disease or a predisposition for a disease selected from the group consisting of diabetes, chronic inflammation, brain edema, arthritis, uvietis, macular edema, hyperglycemia, a kidney inflammatory disease, a disorder resulting in kidney fibrosis, a disorder of the kidney resulting in proteinuria, and combinations thereof.

14. The method of claim 12 wherein the effective amount of the composition causes a statistically significant inhibition of endogenous VEGF expression.

15. The method of claim 12 wherein the effective amount of the composition causes a statistically significant inhibition of endogenous TGF-β expression.

16. A method of inhibiting fibrosis in an animal who is not exhibiting pathological angiogenesis, comprising the step of:
administering to an animal in need thereof an effective amount of a composition capable of inhibiting fibrosis, wherein the composition comprises kallistatin, and wherein the effective amount of the composition is insufficient to inhibit pathological angiogenesis.

17. The method of claim 16 wherein the animal has a disease or a predisposition for a disease selected from the group consisting of diabetes, chronic inflammation, brain edema, arthritis, uvietis, macular edema, hyperglycemia, a kidney inflammatory disease, a disorder resulting in kidney fibrosis, a disorder of the kidney resulting in proteinuria, and combinations thereof.

18. The method of claim 16 wherein the effective amount of the composition causes a statistically significant inhibition of endogenous VEGF expression.

19. The method of claim 16 wherein the effective amount of the composition causes a statistically significant inhibition of endogenous TGF-β expression.

20. A method of inhibiting inflammation in an animal who is not exhibiting pathological angiogenesis, comprising the step of:
administering to an animal in need thereof an effective amount of a composition capable of inhibiting inflammation, wherein the composition comprises kallistatin, and wherein the effective amount of the composition is insufficient to inhibit pathological angiogenesis.

21. The method of claim 20 wherein the animal has a disease or a predisposition for a disease selected from the group consisting of diabetes, chronic inflammation, brain edema, arthritis, uvietis, macular edema, hyperglycemia, a kidney inflammatory disease, a disorder resulting in kidney fibrosis, a disorder of the kidney resulting in proteinuria, and combinations thereof.

22. The method of claim 20 wherein the effective amount of the composition causes a statistically significant inhibition of endogenous VEGF expression.

23. The method of claim 20 wherein the effective amount of the composition causes a statistically significant inhibition of endogenous TGF-β expression.
